(19) United States
(12) Patent Application Publication
(10) Pub. No.: US 2025/0281692 A1
(43) Pub. Date: Sep. 11, 2025

(54) INSERTION DEVICE FOR AN INFUSION SET

(71) Applicant: Ypsomed AG, Burgdorf (CH)

(72) Inventors: Simon Scheurer, Bern (CH); Patrick Hostettler, Hasle (CH)

(21) Appl. No.: 19/220,015

(22) Filed: May 27, 2025

Related U.S. Application Data
(63) Continuation of application No. PCT/EP2023/087396, filed on Dec. 21, 2023.

(30) Foreign Application Priority Data
Dec. 23, 2022 (CH) 001577/2022

(51) Int. Cl. A61M 5/158

(52) U.S. Cl. CPC A61M 5/158; A61M 2005/1585; A61M 2205/8281

(57) ABSTRACT

An insertion device for an infusion set includes an advancing element for moving the infusion set from a proximal position to a distal position along a central axis, a retaining element for retaining the infusion set, and a push button. The push button is displaceable along the central axis by an actuation travel and is configured such that, when the push button is actuated by the actuation travel, the retaining element is moved radially outward to at least partially release the infusion set from the retaining element.

### CROSS-REFERENCE TO RELATED APPLICATIONS

This application is a continuation of International Patent Application No. PCT/EP2023/087396, filed Dec. 21, 2023, entitled “INSERTION DEVICE FOR AN INFUSION SET,” which in turn claims priority to Swiss Patent Application No. CH001577/2022, filed Dec. 23, 2022, entitled “INSERTION DEVICE FOR AN INFUSION SET”, each of which is incorporated by reference herein, in the entirety and for all purposes.

### TECHNICAL FIELD

The present disclosure relates to an insertion device for an infusion set.

### BACKGROUND

Infusion sets are known from WO02/070037A2 and from WO2005/049117A2. Such infusion sets are used for insulin pump therapy where insulin is transported by the insulin pump into the body of a diabetic person with the aid of an infusion set. The infusion set comprises a cannula, which may be placed directly under or into the skin by means of an insertion device or injection aid and is held in place by an adhesive patch.

An insertion device for such an infusion set is known from EP2979713B1. One disadvantage of said insertion device is that the infusion set is pushed out of the device without guidance, resulting in the risk that the cannula does not penetrate sufficiently perpendicularly to the injection site. This can cause skin injuries to the patient.

### SUMMARY

It is therefore an object of the present disclosure to provide easier and safer insertion of an infusion set by means of such an insertion device. This object is achieved by the subject matter of the present disclosure.

According to certain implementations, an insertion device for an infusion set may include an advancing element for moving the infusion set from a proximal position to a distal position along a central axis (z). The insertion device further comprises a retaining element for retaining the infusion set, and a base part having a contact surface for placing the insertion device on an application site on the body of a patient, wherein the base part is received by the retaining element. To retain the infusion set, the retaining element has at least one retaining arm, two or a multiple of two retaining arms. The retaining arm is designed in such a way that a retaining wing provided on the infusion set can be snapped into and out of the retaining arm. The retaining arm may be elastic and/or able to be tensioned. The insertion device further comprises a head part, which is arranged so as to be rotatable relative to the base part about the central axis (z). Upon a relative rotation between the head part and the base part in a first direction of rotation, a guide element attached to the retaining element can be guided by a mating guide element provided on the base part, wherein the retaining element is movable relative to the base part from a distal position to a proximal position along the central axis (z).

Upon a relative rotation between the head part and the base part in a second direction of rotation, the guide element of the retaining element can be guided by the mating guide element provided on the base part, wherein the retaining element is movable relative to the base part from the proximal position to the distal position along the central axis (z). The retaining element is movable along the axis (z) since the guide element of the retaining element is in guiding engagement with the mating guide element provided on the base part.

The guide element of the retaining element may be designed as a guide curve and the mating guide element of the base part is designed as a guide cam. Alternatively, the guide element of the retaining element may be designed as a guide cam and the mating guide element of the base part may be designed as a guide curve. A different guide element or a different mating guide element may also be provided an external thread and a corresponding internal thread, provided that the guide element and the mating guide element are in guiding engagement.

The retaining element may be connected to the head part in such a way as to rotate therewith. The head part can rotate with the retaining element relative to the base part in a first direction of rotation and/or second direction of rotation.

In addition, a cover may be connected to the head part in such a way as to rotate therewith. Alternatively, the cover may be formed in one piece with the head part. The head part may have received the cover at its proximal end.

The advancing element may comprise an elastic element and a sliding element. Upon a relative rotation between the head part and the base part in the first direction of rotation, the elastic element can be tensioned; upon a relative rotation between the head part and the base part in the second direction of rotation, the elastic element can be relaxed. The elastic element may be supported on the sliding element and on the head part or the cover, or alternatively may be supported on the base part. The elastic element may thus be designed as a compression spring or tension spring.

In the proximal position of the advancing element, the sliding element may can be rotated from a first position to a second position, wherein in the first position the sliding element can be retained by the retaining element and in the second position the sliding element can be released by the retaining element such that the sliding element is axially movable relative to the retaining element along the central axis (z).

In the distal position and in the proximal position of the advancing element, the sliding element may be arranged offset relative to the retaining element by an angle of rotation by an angle of rotation of approximately 10°, approximately 12°, or approximately 15°. A different angle of rotation may also be provided.

The sliding element may have a securing surface, a projection or a ramp-like projection. The securing surface or the projection, serves to retain or better retain, the infusion set in the retaining element. In the first position of the sliding element, the securing surface or the projection of the sliding element, may be in abutting contact with the retaining wing of the infusion set in order to pretension the retaining arm of the retaining element radially inward and to retain the infusion set. Thus, in the first position of the sliding element, the snap-in connection between the retaining wing of the infusion set and the retaining arm of the retaining element is secured. The snap-in connection between the retaining element and the infusion set better retains the infusion set in the insertion device. When the insertion device has the infusion set inserted, the patient can remove a covering of the adhesive patch without prematurely releasing the infusion set from the insertion device or damaging the infusion set. The securing surface or the projection of the sliding element, can reach the second position of the sliding element by a rotation relative to the retaining element, wherein the securing surface or the projection of the sliding element, is arranged radially offset relative to the retaining arm. Thus, in the second position of the sliding element, the infusion set can be more easily pushed out of the retaining arm of the retaining element since the infusion set is no longer additionally retained in the retaining arm by way of the sliding element, but rather the infusion set is retained in the retaining element only by way of the snap-in connection between the retaining wing of the infusion set and the retaining arm of the retaining element.

The sliding element may have an annular support for pushing the infusion set out of the retaining element, or may be designed as an annular support. The annular support may be sleeve-like. The annular support may be at least partially ring-shaped. The annular support may come into abutting contact with the infusion set or with a housing or a cap-shaped housing of the infusion set. The infusion set can thus be pushed out of the insertion device in a guided manner.

The cover may have received a push button. The push button may be displaceable along the central axis (z) by an actuation travel distance (H). Furthermore, the push button may be designed such that, when the push button is actuated by the actuation travel distance (H), the sliding element can be rotated relative to the retaining element about the central axis (z) by the angle of rotation, by an angle of rotation of approximately 10°, approximately 12°, or approximately 15°, into the second position.

The push button may be displaceable along the central axis (z) by an actuation travel distance (H) and may be designed such that, when the push button is actuated by the actuation travel distance (H), the retaining element is moved radially outward in order to at least partially release the infusion set from the retaining element. The snap-in connection between the retaining wing of the infusion set and the retaining arm of the retaining element can thus be released.

The retaining element and the push button may each have sliding surfaces, which are designed such that, when the push button is actuated by the actuation travel distance (H), the retaining element is moved radially outward in order to at least partially release the infusion set from the retaining element.

The push button may have a snap-in arm or a snap-in hook on the snap-in arm, which can move the retaining arm of the retaining element radially outward in order to at least partially release the infusion set from the retaining arm. This movement may be a spreading-apart, a pretensioning or a deflection in the radially outward direction. To this end, the snap-in hook on the push button and/or the retaining arm of the retaining element may have sliding surfaces or inclined sliding surfaces. The sliding surfaces may come into sliding contact.

This movement may take place at least partially before and/or during the pushing of the annular support against the infusion set.

Furthermore, the sliding element comprises a cutout for receiving a retaining tab provided on the infusion set. The patient can hold the infusion set at the retaining tab of the infusion set. The retaining tab may be of elongate shape. However, other shapes may also be provided. The cutout of the sliding element is designed in such a way that the retaining tab can be inserted in one orientation or in two orientations, wherein these two orientations are offset from each other by 180° in the circumferential direction. Easy insertion of the infusion set into the insertion device can thus be ensured.

### BRIEF DESCRIPTION OF THE DRAWINGS

Implementations are described below with reference to the drawings. Both individually and in combination, the features disclosed herein advantageously further develop disclosed implementations. In the drawings:

FIGS. 1a and 1b show exploded views of an embodiment of an insertion device according to the present disclosure.

FIG. 2 shows a longitudinal cross-sectional view of the embodiment of the insertion device according to FIG. 1a or 1b in a starting position, the longitudinal cross-sectional view corresponding to the section line A-A indicated in FIG. 1a or in FIG. 1b.

FIG. 3 shows a longitudinal cross-sectional view of the embodiment of the insertion device according to FIG. 1a or 1b with an infusion set inserted, the longitudinal cross-sectional view corresponding to the section line A-A indicated in FIG. 1a or in FIG. 1b.

FIG. 4 shows a longitudinal cross-sectional view of the embodiment of the insertion device according to FIG. 1a or 1b in a loaded position, the longitudinal cross-sectional view corresponding to the section line A-A indicated in FIG. 1a or in FIG. 1b.

FIG. 5 shows a longitudinal cross-sectional view of the embodiment of the insertion device according to FIG. 1a or 1b in a released position, the longitudinal cross-sectional view corresponding to the section line A-A indicated in FIG. 1a or in FIG. 1b.

FIG. 6 shows a longitudinal cross-sectional view of the embodiment of the insertion device according to FIG. 1a or 1b with the infusion set pierced into the skin of a patient, the longitudinal cross-sectional view corresponding to the section line A-A indicated in FIG. 1a or in FIG. 1b.

### DETAILED DESCRIPTION

### Definitions

Hereinbelow, “proximal position of the insertion device” means a position in which an advancing element for advancing the infusion set into the skin is in a tensioned state, and “distal position of the insertion device” means a position in which the advancing element for advancing the infusion set into the skin is in a relaxed state.

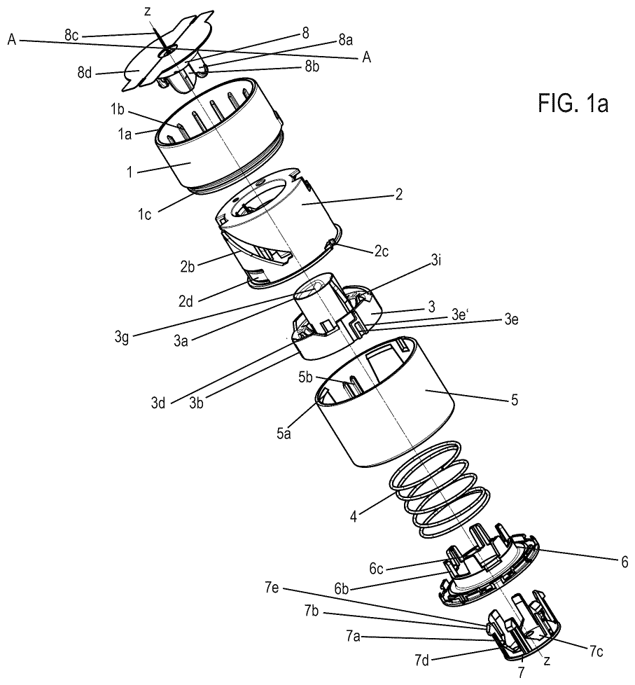

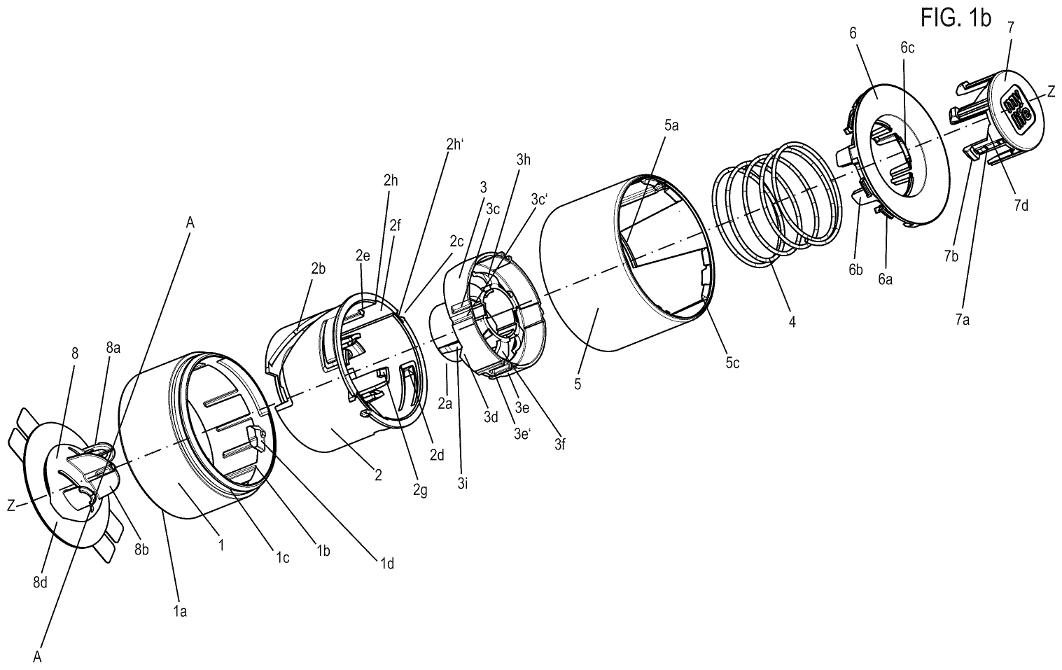

FIGS. 1a and 1b show exploded views of one embodiment of an insertion device according to the present disclosure. The injection device comprises a sliding element (3) and a retaining element (2) for retaining an infusion set (8), wherein the infusion set (8) may comprise a cannula (8c) for piercing into the skin of a patient and an adhesive patch (8d) for securing the infusion set to the patient's skin. To this end, the retaining element (2) comprises at least two retaining arms (2a), wherein the at least two elastic retaining arms (2a) are arranged offset from each other by 180° in the circumferential direction. The retaining arms (2a) are designed in such a way that two retaining wings (8a) arranged on the infusion set (8) can snap into and out of the retaining arms (2a). To enable the infusion set (8) to be inserted into the insertion device, the sliding element (3) has a cutout (3g) at the distal end. A retaining tab (2b) attached to the infusion set (8) can be received in the cutout (3g). The insertion device further comprises a head part (5) and a base part (1), wherein the base part (1) has received the retaining element (2). The base part (1) comprises a contact surface (1a) for placing the insertion device on an application site on the body of the patient. Longitudinal webs (1b) for axially guiding the retaining element (2) along a central axis (z) are arranged on the inner lateral surface of the base part (1). An annular groove (lc) is provided at the proximal end and on the outer lateral surface of the base part (1). A snap fastener (5a) is attached to the distal end and on the inner lateral surface of the head part (5). The snap fastener (5a) of the head part (5) is snapped into the annular groove (lc) of the base part (1) such that the head part (5) is rotatable relative to the base part (1). The retaining element (1) has a guide element in the form of a guide curve (2b), which is in engagement with a mating guide element in the form of a guide cam (1d) provided on the base part (1). Upon a relative rotation between the head part (5) and the base part (1) in a first direction of rotation, the guide curve (2b) of the retaining element (2) can be guided by the guide cam (1d) provided on the base part (1), wherein the retaining element (2) is movable relative to the base part (1) from a distal position to a proximal position along the central axis (z). The retaining element (2) can be guided due to or by an element of the guide pair (1d; 2b). Upon a relative rotation between the head part (5) and the base part (1) in a second direction of rotation, the guide curve (2b) of the retaining element (2) can likewise be guided by the guide cam (1d) provided on the base part (1), wherein the retaining element (2) is movable relative to the base part (1) from the proximal position to the distal position along the central axis (z). The retaining element (2) is connected to the head part (5) in an axially movable manner and in such a way as to rotate therewith. To this end, the retaining element (2) comprises a rib (2c) on the outer lateral surface, which rib is in engagement with a groove (5b) provided on the inner lateral surface of the head part (5). The insertion device further comprises an advancing element. The advancing element comprises an elastic element in the form of a compression spring (4) and the sliding element (3). The compression spring (4) is mounted between the sliding element (3) and a cover (6) in such a way as to be able to be tensioned. The cover (6) is connected to the head part (5) in an axially fixed manner by way of a snap-in connection. To this end, the cover (6) has at its proximal end a cover groove (6a), which is in engagement with an annular web (5c) provided on the head part (5). The sliding element (3) has an annular support (3a) in order to push the infusion set (8) out of the insertion device or to push it out in a guided manner. Also provided on the sliding element (3) is a sliding element stop (3b), which can come into abutting contact with a retaining element stop (2e) attached to the inner lateral surface of the retaining element (2). By virtue of the abutting contact between the sliding element stop (3b) and the retaining element stop (2e), the sliding element (3) is connected to the retaining element (2) in an axially fixed manner. To prevent the sliding element (3) from coming out of abutting contact with the retaining element (2) of its own accord, a snap fastener (2d) provided on the inner lateral surface of the retaining element (2) can snap into a first channel (3c) provided on the outer lateral surface of the sliding element (3). Abutting contact may additionally be provided between a first sliding element web (3e) provided on the sliding element (3) and a first retaining element web (2h) attached to the retaining element (2), in order to limit a relative rotation between the sliding element (3) and the retaining element (2) in a first direction of rotation. In order to limit a relative rotation between the sliding element (3) and the retaining element (2) in a second direction of rotation, abutting contact is provided between a second sliding element web (3e′) provided on the sliding element (3) and a second retaining element web (2h′) attached to the retaining element (2). When the second sliding element web (3e′) is in abutting contact with the second retaining element web (2h′), the sliding element (3) is out of abutting contact with the retaining element (2), so that the sliding element (3) is axially displaceable relative to the retaining element (2). To this end, the retaining element (2) has a guide groove (2f) on the inner lateral surface, wherein the sliding element stop (3b) can slide with the first and second sliding element webs (3e, 3e′) along the guide groove (2f). During this, the snap fastener (2d) of the retaining element (2) may lie in a second channel (3c′) of the sliding element (3), wherein the second channel (3c′) is offset from the first channel (3c) of the sliding element in the circumferential direction. The sliding element (3) further comprises a control curve (3d), which can come into guiding engagement with the guide cam (1d) of the base part (1). Upon a relative rotation of the head part (5) and the base part (1), the sliding element (3) can be rotated relative to the retaining element (2) by way of the guide cam (1d) of the base part (1) and the control curve (3d) of the sliding element (3). The sliding element (3) moves relative to the retaining element (2) from a first to a second position. The guide curve (2b) of the retaining element (2), the guide cam (1d) of the base part (1) and the control curve (3d) of the sliding element (3) are designed and arranged in such a way that the sliding element (3) can be rotated relative to the retaining element (2). The angle of rotation between the sliding element (3) and the retaining element (2) may be approximately 10°. Different angles of rotation may also be provided approximately 12° or 15°. The insertion device further comprises a push button (7), which is received by the cover (6). The push button (7) has a snap-in arm (7a) projecting in the distal direction. The snap-in arms (7a) are in sliding contact with cover webs (6b) provided on the cover (6). A snap-in hook (7b) attached to the respective snap-in arm (7a) forms a stop with the distal end of the cover web (6b), so that the relative axial movement in the proximal direction of the push button (7) relative to the cover (6) is limited. The snap-in arm (7a) or the snap-in hook (7b) of the snap-in arm (7a) of the push button, is displaceable along the central axis (z) by an actuation travel distance (H) and is designed such that, when the push button (7) is actuated by the actuation travel distance (H), the retaining element (2) is moved radially outward in order to at least partially release the infusion set (8) from the retaining element (2). A sliding surface (7e) or an inclined sliding surface (7e), is provided on the snap-in hook (7b) of the push button (7) and can come into sliding contact with a sliding surface (2g or an inclined sliding surface (2g), provided on the snap-in arm (2a) of the retaining element (2), wherein, when the push button is actuated by the actuation travel distance (H), the snap-in arm (2a) of the retaining element (2) is moved radially outward in order to at least partially release the infusion set (8) or the retaining wing (8a) of the infusion set (8), from the retaining arm (2a) of the retaining element. The relative axial movement in the distal direction of the push button (7) relative to the cover (6) is formed between a push button disk (7c) provided at the proximal end of the push button (7) and a cover shoulder (6c) attached to the proximal end of the cover (6). The push button (7) further comprises a push button bevel (7d), which can interact with a sliding element edge (3h) of the sliding element (3) provided at a sliding element opening (3f) in order to rotate the sliding element (3) relative to the retaining element (2).

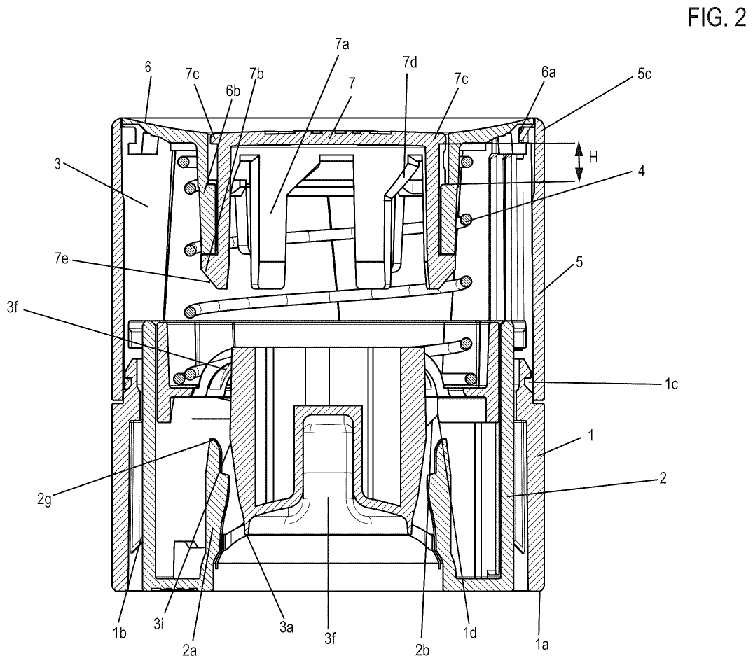

FIG. 2 shows a longitudinal cross-sectional view of the embodiment of the insertion device according to FIG. 1a or 1b in a starting position, the longitudinal cross-sectional view corresponding to the section line A-A indicated in FIG. 1a or FIG. 1b. The retaining element (2), the push button (7) and the advancing element (3, 4) are in the distal position, with no infusion set yet having been inserted in the insertion device. The guide cam (1d) of the base part (1) is in abutting contact with the proximal end of the control curve (2b) of the retaining element (2). The sliding element stop (3b) of the sliding element bears against the retaining element stop (2e) of the retaining element (2) such that the sliding element (3) is connected to the retaining element (2) in an axially fixed manner. The snap fastener (2d) of the retaining element (2) projects into the first channel (3c) of the sliding element (3) such that the sliding element (3) cannot come out of contact with the retaining element (2) of its own accord. The compression spring (4) is mounted between the cover (6) and the sliding element (3) such that the sliding element (3) and the retaining element (2) are held in the distal position. The compression spring (4) may be relaxed or pretensioned.

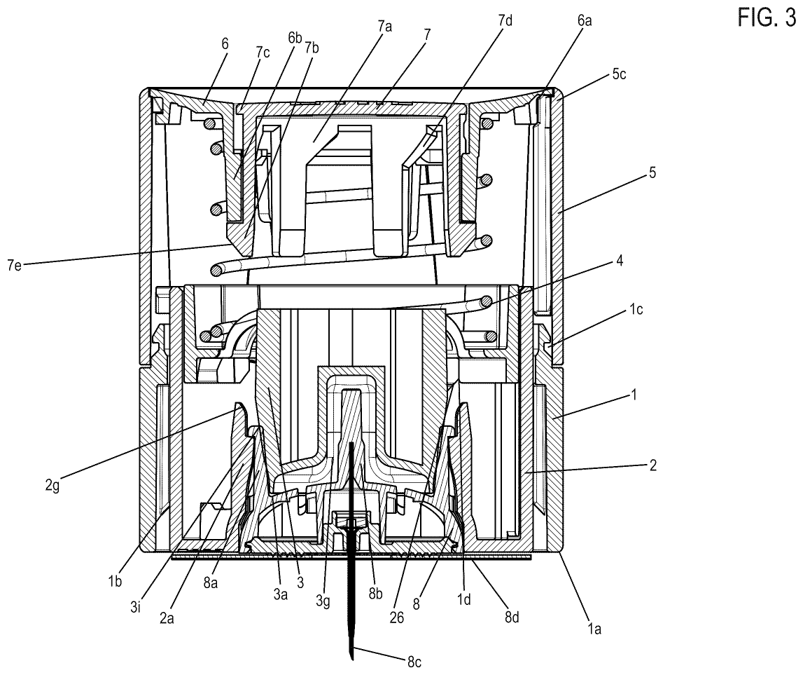

FIG. 3 shows a longitudinal sectional view of the embodiment of the insertion device according to FIG. 1a or 1b with the infusion set inserted, the longitudinal sectional view corresponding to the section line A-A indicated in FIG. 1a or FIG. 1b. Using the retaining tab (8b), the patient guides the infusion set (8) through the cutout (3g) of the sliding element (3). The cutout (3g) of the sliding element (3) is designed in such a way that the retaining tab (8b) can be inserted in two orientations, which are offset from each other by 180° in the circumferential direction. The retaining wings (8a) of the infusion set (8) snap into the retaining arms (8a) of the retaining element (2). The infusion set (8) is inserted into the insertion device. The sliding element (3 or the annular support (3a) of the sliding element (3), and the infusion set (8) are located at a distance from each other along the central axis (z). In the first position, a projection (3i) provided on the sliding element is in abutting contact with the retaining wing (8a) of the infusion set in order to hold the infusion set (8) or retaining wing (8a) of the infusion set (8), in the retaining arm (2a). The projection (3i) of the sliding element (3) tensions the retaining arm (2a) of the retaining element (2) in a radially outward direction. The projection (3i) of the sliding element (3) is ramp-like.

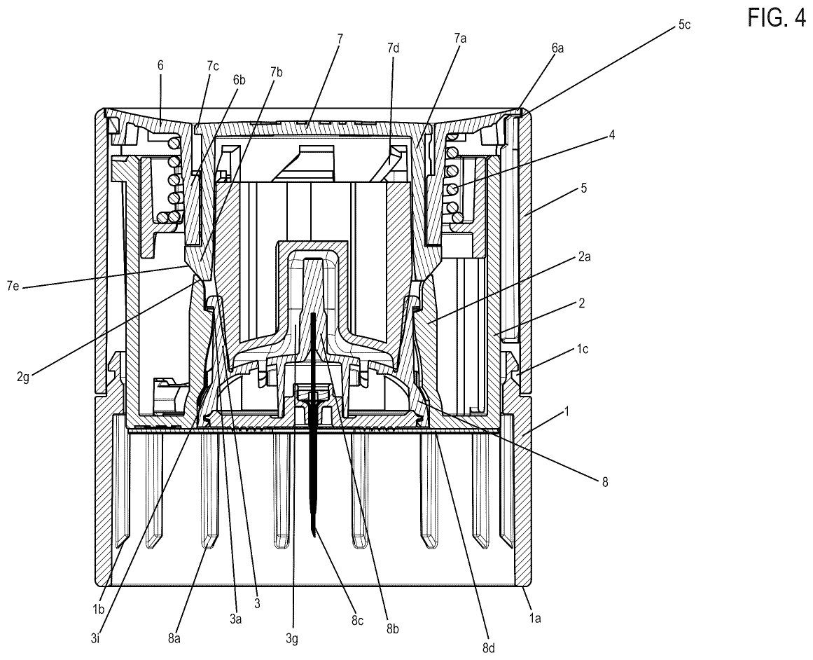

FIG. 4 shows a longitudinal cross-sectional view of the embodiment of the insertion device according to FIG. 1a or 1b in a loaded position, the longitudinal cross-sectional view corresponding to the section line A-A indicated in FIG. 1a or FIG. 1b. The patient rotates the head part (5) relative to the base part (1) of the insertion device in the first direction of rotation. The guide curve (2b) of the retaining element (2) is guided by the guide cam (1d) arranged on the base part (1) such that the retaining element (2) is moved relative to the base part (1) from the distal position to the proximal position along the central axis (z). The guide cam (1d) of the base part (1) comes to lie in abutting contact with the distal end of the control curve (2b) of the retaining element (2). The distal end of the control curve (2b) of the retaining element (2) and the guide cam (1d) of the base part (1) are designed in such a way that the guide cam (1d) of the base part (1) can hold the retaining element (2) in the proximal position. The sliding element stop (3b) of the sliding element (3) bears against the retaining element stop (2e) of the retaining element (2) such that the sliding element (3) is moved with the retaining element (2) into the proximal position. The spring (4), which is mounted between the sliding element (3) and the cover (6), is thereby tensioned or further tensioned.

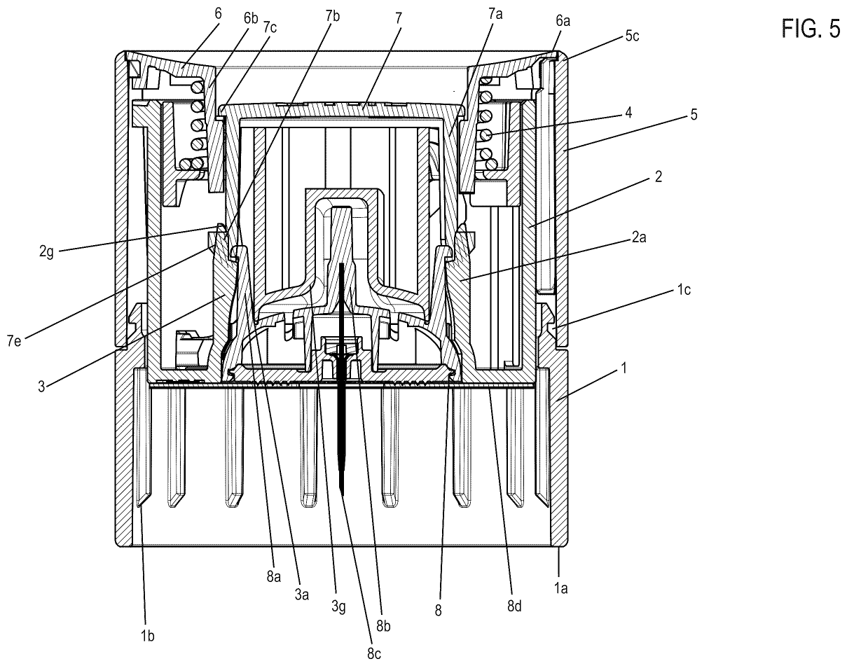

FIG. 5 shows a longitudinal cross-sectional view of the embodiment of the insertion device according to FIG. 1a or 1b in a released position, the longitudinal cross-sectional view corresponding to the section line A-A indicated in FIG. 1a or FIG. 1b. The patient pushes the push button (7) in the distal direction. The push button bevel (7d) of the push button (7) interacts with the sliding element edge (3h) of the sliding element opening (3f) of the sliding element (3) such that the sliding element (3) is rotated relative to the retaining element (2), by the angle of rotation of approximately 10°. The projection (3i) provided on the sliding element is in the second position and thus out of abutting contact with the retaining wing (8a) of the infusion set (8). The retaining arm (2a) of the retaining element (2) is no longer pretensioned in the radially outward direction by the projection (3i) of the sliding element (3). The retaining arm (2a) of the retaining element (2) is relaxed. When the insertion device is released, the snap fastener (2d) of the retaining element (2) comes to lie in the second channel (3c′) of the sliding element (3). The sliding element stop (3b) of the sliding element (3) comes out of abutting contact with the retaining element stop (2e) of the retaining element (2). The first and the second sliding element web (3e, 3e′) of the sliding element are in guiding engagement with the guide groove (2f) of the retaining element (2) such that the sliding element (3) can be moved relative to the retaining element (2) by way of the spring force of the tensioned spring (4). The annular support (3a) of the sliding element (3) pushes against the infusion set (8), or against a cap-shaped housing of the infusion set, with the aid of the spring force of the spring (4) such that the retaining wings (8a) of the infusion set (8) snap out of the retaining arms (2a) of the retaining element (2) and the infusion set (8) is moved distally. In addition, the retaining arm (2a) of the retaining element (2) and the snap-in hook (7b) of the push button (3) each have sliding surfaces (2g; 7e), which are designed such that, when the push button (7) is actuated by the actuation travel distance (H), the retaining arm (2a) of the retaining element (2) is moved radially outward in order to at least partially release the infusion set (8) or the retaining wing (8a) of the infusion set (8), from the retaining element (2) or the retaining arm (2a) of the retaining element (2). The infusion set with the cannula (8c) provided on the infusion set can be pierced into the patient's skin.

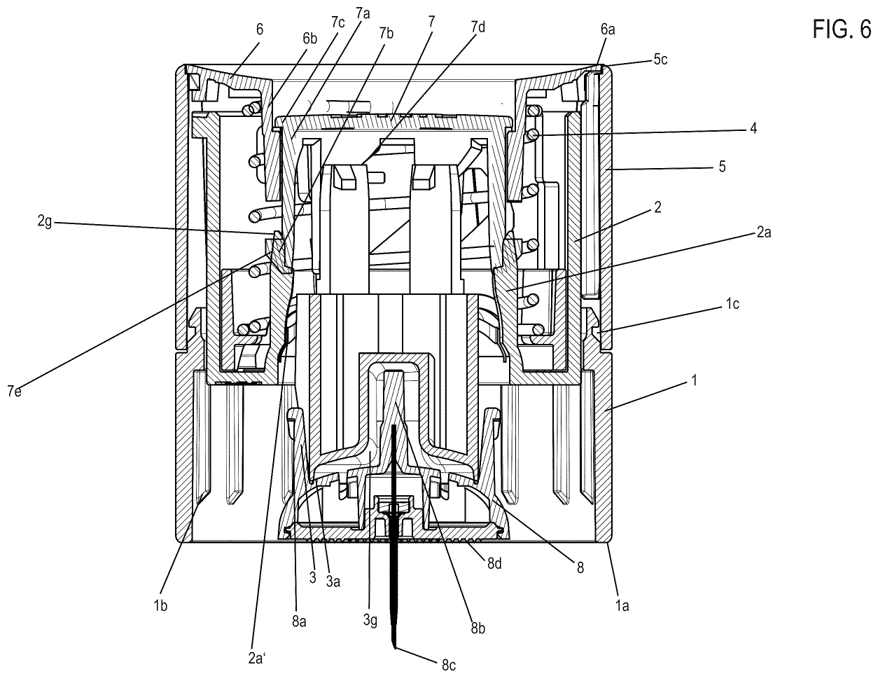

FIG. 6 shows a longitudinal cross-sectional view of the embodiment of the insertion device according to FIG. 1a or 1b with an infusion set pierced into the skin of a patient, the longitudinal cross-sectional view corresponding to the section line A-A indicated in FIG. 1a or FIG. 1b. The cannula (8c) of the infusion set (8) is pierced into the patient's skin. The push button disk (7c) of the push button (7) is in abutting contact with the cover shoulder (6c) of the cover (6). The sliding element (3) comes into abutting contact with the guide cam (1d) of the base part (1). The annular support (3a) of the sliding element (3) is in abutting contact with the infusion set (8) or with the cap-shaped housing of the infusion set (8). The spring (4), which is mounted between the cover (6) and the sliding element (3), is relaxed. The insertion device can then be removed from the patient's skin, with the infusion set (8) remaining on the patient's skin by way of the adhesive patch (8d) provided on the infusion set.

In order to return the insertion device to the starting position shown in FIG. 2, the patient rotates the head part (5) relative to the base part (1) of the insertion device in the second direction of rotation. The guide curve (2b) of the retaining element (2) is guided by the guide cam (1d) arranged on the base part (1) such that the retaining element (2) is moved relative to the base part (1) from the proximal position to the distal position along the central axis (z). The guide cam (l d) of the base part (1) comes into abutting contact with the proximal end of the guide curve (2b) of the retaining element (2). The proximal end of the guide curve (2b) of the retaining element (2), the guide cam (1d) of the base part (1) and the control curve (3d) of the sliding element (3) are designed and arranged in such a way that the sliding element (3) can be rotated relative to the retaining element (2), by the angle of rotation of approximately 10°. The sliding element (3) entrains the push button (7) by way of the sliding element edge (not visible) of the sliding element (3) and the push button bevel (7d) of the push button. The snap-in hook (7b) of the push button (7) comes into abutting contact with the distal end of the cover web (6b) of the cover (6). The retaining element (2) is in the distal position, so that a new infusion set (8) can be inserted into the insertion device.

Various changes may be made in the form, construction and arrangement of the components of the present disclosure without departing from the disclosed subject matter. Moreover, the embodiments are illustrative and that the scope of the disclosure is not limited to them. Many variations, modifications, additions, and improvements are possible. These and other variations, modifications, additions, and improvements may fall within the scope of the disclosure as defined in the claims that follow.

### REFERENCE SIGNS

1 base part
1a contact surface
1b longitudinal web
1c annular groove
1d guide cam
2 retaining element
2a retaining arm
2b guide curve
2c rib
2d snap fastener
2e retaining element stop
2f guide groove
2g sliding surface
2h first retaining element web
2h′ second retaining element web
3 sliding element
3a annular support
3b sliding element stop
3c first channel
3c′ second channel
3d control curve
3e first sliding element web
3e′ second sliding element web
3f sliding element opening
3g cutout
3h sliding element edge
3i securing surface, projection
4 compression spring
5 head part
5a snap fastener
5b longitudinal groove
5c annular web
6 cover
6a cover groove
6b cover web
6c cover shoulder
7 push button
7a snap-in arm
7b snap-in hook
7c push button disk
7d push button bevel
7e sliding surface
8 infusion set
8a retaining wing
8b retaining tab
8c cannula
8d adhesive patch
z central axis
A-A section axis
H actuation travel distance

What is claimed is:

1. An insertion device for an infusion set, comprising:
an advancing element configured for moving the infusion set from a proximal position to a distal position along a central axis of the insertion device;
a retaining element configured for retaining the infusion set;
a base part configured to receive the retaining element and comprising a contact surface for placing the insertion device on a patient; and
a head part, wherein the head part and the base part are configured to be rotatable relative to each other about the central axis, wherein the retaining element is coupled to the head part in an axially movable manner and rotatable therewith, and wherein a push button is received at a proximal end of the head part,
wherein a guide element of the retaining element is configured to be in a guiding engagement with a mating guide element of the base part such that, upon a relative rotation between the head part and the base part in a first direction of rotation, the retaining element is movable relative to the base part from a distal position to a proximal position along the central axis, and such that, upon a relative rotation between the head part and the base part in a second direction of rotation opposite from the first direction of rotation, the retaining element is movable relative to the base part from the proximal position to the distal position along the central axis, and
wherein the push button is configured to be displaceable along the central axis by an actuation travel distance such that, when the push button is actuated by the actuation travel distance, the retaining element is moved radially outward to at least partially release the infusion set from the retaining element.

2. The insertion device according to claim 1, wherein the retaining element and the push button each comprise sliding surfaces configured to move the retaining element radially outward to at least partially release the infusion set from the retaining element when the push button is actuated by the actuation travel distance.

3. The insertion device according to claim 2, wherein the sliding surfaces are inclined.

4. The insertion device according to claim 1, wherein the retaining element comprises a retaining arm for retaining the infusion set, and wherein the retaining arm is movable radially outward.

5. The insertion device according to claim 4, wherein a sliding surface or an inclined sliding surface is provided on the retaining arm configured to move the retaining element radially outward to at least partially release the infusion set from the retaining element when the push button is actuated by the actuation travel distance.

6. The insertion device according to claim 1, wherein the push button comprises a snap-in arm with a snap-in hook, wherein a sliding surface is provided on the snap-in hook configured to move the retaining element radially outward to at least partially release the infusion set from the retaining element when the push button is actuated by the actuation travel distance.

7. The insertion device according to claim 6, wherein the sliding surface is configured as an inclined sliding surface.

8. The insertion device according to claim 1, wherein the advancing element comprises an elastic element and a sliding element.

9. The insertion device according to claim 8, wherein the sliding element comprises a securing surface or a projection configured for retaining the infusion set in the retaining element.

10. The insertion device according to claim 8, wherein sliding element comprises an annular support configured for pushing the infusion set out of the retaining element.

11. The insertion device according to claim 8, wherein the elastic element is configured as a compression spring.

12. The insertion device according to claim 8, wherein the sliding element comprises a cutout for receiving a retaining tab provided on the infusion set.

13. The insertion device according to claim 1, wherein the guide element is configured as a guide curve and the mating guide element is configured as a guide cam.
